(19) United States
(12) Patent Application Publication
(10) Pub. No.: US 2003/0078205 A1
(43) Pub. Date: Apr. 24, 2003

(54) Methods and compositions for treating and preventing distal bowel lesions

(76) Inventor: Daniel K. Podolsky, Wellesley, MA (US)

Correspondence Address: CLARK & ELBING LLP, 101 FEDERAL STREET, BOSTON, MA, 02110, US

(21) Appl. No.: 10/208,968

(22) Filed: Jul. 31, 2002

Related U.S. Application Data
(60) Provisional application No. 60/309,238, filed on Jul. 31, 2001.

(51) Int. Cl. A61K 38/17

(52) U.S. Cl. 514/12

(57) ABSTRACT

This invention features compositions and methods for treating and preventing distal bowel disease by rectal administration of intestinal trefoil factors. The intestinal trefoil factor can be administered either alone or in combination with one or more antimicrobial agents, anti-inflammatory agents or analgesics.

### CROSS REFERENCE TO RELATED APPLICATIONS

This application claims benefit of the filing date of U.S. Provisional Application No. 60/309,238 (filed Jul. 31, 2001), hereby incorporated by reference.

### BACKGROUND OF THE INVENTION

The invention features methods for treating lesions of the distal bowel using intestinal trefoil peptides.

Proctitis and enteritis involve the destruction of the small and/or large bowel epithelium, resulting in erythema, ulcerations, diarrhea, bleeding from the bowel, malabsorption of nutrients and, frequently severe abdominal pain. Enteritis and proctitis often arise as a complication of antineoplastic therapy, such as cancer chemotherapy or radiotherapy directed at the abdomen or pelvic area. This syndrome may also result from natural diseases like Crohn's disease, ulcerative colitis, vascular insufficiency, infection, allergic conditions, or from other causes that are less well understood. The painful ulcerative lesions of enteritis or proctitis can cause patients to restrict their food and liquid intake, resulting in weight loss and dehydration. Severe enteritis can necessitate the de-escalation or the complete interruption of a planned chemo/radio-therapeutic dosing.

Secondary infections by pathogenic microorganisms are a serious consequence of the mucosal damage caused by enteritis and proctitis. These conditions, when severe, are risk factors for chronic debilitating local infections (e.g., yeast (Candida spp.) infections) and septicemias. The microorganisms use the compromised bowel epithelium as a portal of entry into the body. The problem of secondary infection is made more serious by the immunocompromised status of patients undergoing cancer treatment (chemotherapy or radiotherapy).

The overall frequency of enteritis and proctitis varies with the patient's diagnosis, age, and general level of health, as well as the type, dose, and frequency of cancer therapy. Approximately 40% of all patients who receive chemotherapy suffer some degree of mucositis of the gastrointestinal tract. Enteritis or proctitis occur in the majority of patients undergoing either total abdominal radiation or high dose radiotherapy to the perineal area.

Vascular, particularly arterial, insufficiency of the bowel is a relatively common and serious problem in patients suffering from atherosclerotic disease, but may also occur in disease-free patients. Reduced blood flow to the gastrointestinal tract can result in epithelial cell loss, making the patient susceptible to malabsorptive conditions and secondary microbial infections.

### SUMMARY OF THE INVENTION

This invention features methods and compositions for treating or preventing a lesion of the distal bowel in a mammal by administering, to the rectum of the mammal, a therapeutically effect amount of an intestinal trefoil peptide. Treatment or prevention of lesions according to the invention can speed healing, reduce pain, delay or prevent occurrence of the lesion, and inhibit expansion, secondary infection, or other complications of the lesion. Preferably, the mammal is a human. In particularly useful embodiments, the intestinal trefoil peptide is SP, pS2, ITF, ITF₁₅₋₇₃, ITF₂₁₋₇₃, ITF₁₋₇₂, ITF₁₅₋₇₂, or ITF₂₁₋₇₂, and is present in a pharmaceutical composition containing a pharmaceutically acceptable carrier. Other useful intestinal trefoil peptides include polypeptides that are substantially identical to SP, pS2, ITF, ITF₁₅₋₇₃, ITF₂₁₋₇₃, ITF₁₅₋₇₂, or ITF₂₁₋₇₂. Preferably, the intestinal trefoil peptide is ITF, which may be administered as a monomer, a dimer, or another multimeric form.

The methods and compositions of this invention are particularly useful for treating lesions of the distal bowel such as proctitis, enteritis, Crohn's Disease, ulcerative colitis, lesions caused by physical trauma or surgical intervention (e.g., biopsy, resection, or hemorrhoidectomy), or lesions caused by antineoplastic therapy (e.g., chemotherapy or radiation therapy). Additionally, lesions of the distal bowel that result from microbial (e.g., bacterial, viral, or fungal) infection are also amenable to treatment.

In preferred embodiments of the methods and compositions, a second therapeutic agent is included. Desirable second therapeutic agents include anti-inflammatory agents, antibacterial agents (e.g., penicillins, cephalosporins, tetracyclines, or aminoglycosides), antifungal agents (e.g., nystatin or amphotericin B), antiviral agents (e.g., acyclovir), analgesics (e.g., lidocaine or benzocaine), or steroids (e.g., triamcinolone, budesonide, or hydrocortisone). Other particularly useful second therapeutic agents include 5-aminosalicylic acid derivatives such as sulfasalazine, mesalamine, olsalazine, and balsalazide, anti-TNF-α monoclonal antibodies such as infliximab (Remicade®), and other drugs useful for treating lesions of the distal bowel such as metronidazole. The second therapeutic agent may be administered within (either before or after administration of the intestinal trefoil peptide) 14 days, 7 days, 1 day, 12 hours, 1 hour, or simultaneously with the intestinal trefoil peptide.

The second therapeutic agent can be present in the same or different pharmaceutical compositions as the intestinal trefoil peptide. When the second therapeutic agent is present in a different pharmaceutical composition, different routes of administration may be used. For example, the second therapeutic may be administered orally, or by intravenous, intramuscular, or subcutaneous injection. The second therapeutic need not be administered rectally.

Suitable pharmaceutical compositions include at least an intestinal trefoil peptide and a pharmaceutically acceptable carrier. Particularly useful pharmaceutical compositions contain bioerodable microspheres that encapsulate one or more of the therapeutic agents. In other useful embodiments, a mucoadhesive or viscosity-enhancing agent is present. Alternatively, the trefoil peptide can be formulated for topical application as a concentrated paste to be applied directly to the lesion via a pledget and stick applicator. Rectal administration of the trefoil peptide may be supplemented by oral administration the same or a different trefoil peptide.

Mammalian intestinal trefoil peptides were discovered in 1982. One of the mammalian intestinal trefoil peptides, human intestinal trefoil factor (ITF; TFF3), has been characterized extensively, and is described in U.S. Pat. Nos. 6,063,755, and 6,221,840, hereby incorporated by reference. The other two known human intestinal trefoil peptides are spasmolytic polypeptide (SP; TFF2) and pS2 (TFF1). Intestinal trefoil peptides, described extensively in the literature (e.g., Sands et al., Annu. Rev. Physiol. 58: 253-273, 1996), are expressed in the gastrointestinal tract and have a three-loop structure formed by intrachain disulfide bonds between conserved cysteine residues. These peptides protect the intestinal tract from injury and can be used to treat intestinal tract disorders such as peptic ulcers and inflammatory bowel disease. Homologs of these human peptides have been found in a number of non-human animal species. All members of this protein family, both human and non-human, are referred to herein as intestinal trefoil peptides. Human ITF will be referred to most extensively in this application; however, the activity of human ITF is common to each of the mammalian intestinal trefoil peptides.

“Intestinal trefoil peptide,” as used herein, includes all mammalian homologs of human spasmolytic polypeptide (SP; also known as TFF2), human pS2 (also known as TFF1) and human intestinal trefoil factor (ITF; also known as TFF3), and biologically active fragments thereof. Homologs of the trefoil peptides have, preferably, 70% amino acid identity to the human sequence, more preferably 85% identity, most preferably 95%, or even 99% sequence identity. The length of comparison sequences will generally be at least about 10 amino acid residues, usually at least 20 amino acid residues, more usually at least 30 amino acid residues, typically at least 45 amino acid residues, and preferably more than 60 amino acid residues. Alternatively, intestinal trefoil peptides are polypeptides encoded by a polynucleotide that hybridizes with high stringency to the human ITF, pS2, or SP cDNAs provided in SEQ ID NOs: 4, 5, and 6, respectively, or the human ITF, pS2, or SP genes provided in SEQ ID NOs: 7, 8, and 9, respectively.

The term “fragment” is meant to include polypeptides that are truncations or deletions of SP, pS2 and ITF. Preferably, the fragments have 70% amino acid identity to the corresponding regions of the human polypeptide sequence. More preferably, the fragments are 85% identical, most preferably 95%, or even 99% identical to the human polypeptide sequence to which they correspond. The length of comparison sequences will generally be at least about 10 amino acid residues, usually at least 20 amino acid residues, more usually at least 30 amino acid residues, typically at least 45 amino acid residues, and preferably more than 60 amino acid residues.

Preferable fragments contain four cysteine residues in any positions which correspond to the cysteines at positions 25, 35, 45, 50, 51, 62, or 71, of human ITF (FIG. 1), or positions 31, 41, 51, 56, 57, 68, and 82 of human pS2 (FIG. 2). More preferably, fragments contain five cysteine residues at these positions. Most preferably, six, or even all seven cysteines are present.

Fragments of SP are meant to include truncations or deletions and preferably have 70% sequence identity to the corresponding human SP polypeptide sequence (FIG. 3). More preferably, the fragments are 85% identical, most preferably 95%, or even 99% identical to the human polypeptide sequence. Preferably, active fragments contain at least four cysteine residues which correspond to positions 6, 8, 19, 29, 34, 35, 46, 58, 68, 78, 83, 84, 95, and 104 in the human SP polypeptide. More preferably, fragments contain six cysteines which correspond to these positions. Even more preferable are fragments that contain eight cysteines. Most preferable are fragments that contain cysteines at ten, twelve, or even, all fourteen positions.

It is recognized in the art that one function of the identified cysteine residues is to impart the characteristic three-loop (trefoil) structure to the protein. Accordingly, preferred fragments of ITF and pS2 have a least one loop structure, more preferably, the fragments have two loop structures, and most preferably, they have three loop structures. It is equally well recognized that the native SP polypeptide has a six loop confirmation. Preferable fragments contain at least two of these loop structures, more preferably, four loop structures are conserved, and most preferably, five, or even all six loop structures are present.

By “co-formulated” is meant any single pharmaceutical composition which contains two or more therapeutic or biologically active agents.

By “pharmaceutical preparation” or “pharmaceutical composition” is meant any composition which contains at least one therapeutically or biologically active agent and is suitable for administration to a patient. For the purposes of this invention, pharmaceutical compositions suitable for delivering a therapeutic to the distal bowel include, but are not limited to suppositories, enemas, and pastes. Any of these formulations can be prepared by well known and accepted methods of art. See, for example, Remingtion: The Science and Practice of Pharmacy, 19^{th} edition, (ed. AR Gennaro), Mack Publishing Co., Easton, Pa., 1995.

By “therapeutically effective amount” is meant an amount sufficient to provide medical benefit. When administering trefoil peptides to a human patient according to the methods described herein, a therapeutically effective amount is usually about 1-2500 mg of intestinal trefoil peptide per dose. Preferably, the patient receives, 10 mg, 100 mg, 500 mg, 750 mg, 1000 mg, 1500 mg, or 2000 mg of intestinal trefoil peptide in each dose. Dosing is typically performed 1-5 times each day.

By “distal bowel” is meant the portion of the digestive system that includes the ascending, transverse, descending, and sigmoid colon, rectum, and anal sphincter.

By “biologically active,” when referring to an intestinal trefoil peptide, fragment, or homolog is meant any polypeptide that exhibits an activity common to its related, naturally occurring family member, and that the activity is common to the family of naturally occurring intestinal trefoil peptides. An example of a biological activity common to the family of trefoil peptides is the ability to alter gastrointestinal motility in a mammal.

By “isolated DNA” is meant DNA that is free of the genes which, in the naturally-occurring genome of the organism from which the given DNA is derived, flank the DNA. Thus, the term “isolated DNA” encompasses, for example, cDNA, cloned genomic DNA, and synthetic DNA.

By “treating” is meant administering a pharmaceutical composition for prophylactic and/or therapeutic purposes. The active ingredients of the pharmaceutical composition can treat the primary indication (e.g., epithelial lesion) or secondary symptoms (e.g., concomitant infection, pain, or inflammation).

By “analgesic” is meant an agent which relieves pain by elevating the pain threshold without significantly disturbing the consciousness of the patient.

By “antimicrobial agent” is meant any compound that alters the growth of bacteria or fungi cells, or viruses whereby growth is prevented, stabilized, or inhibited, or wherein the microbes are killed. In other words, the antimicrobial agents can be microbiocidal or microbiostatic.

By “antineoplastic therapy” is meant any treatment regimen used to treat cancer. Typical antineoplastic therapies include chemotherapy and radiation therapy.

By “substantially identical” is meant a polypeptide or nucleic acid exhibiting at least 75%, but preferably 85%, more preferably 90%, most preferably 95%, or 99% identity to a reference amino acid or nucleic acid sequence. For polypeptides, the length of comparison sequences will generally be at least 20 amino acids, preferably at least 30 amino acids, more preferably at least 40 amino acids, and most preferably 50 amino acids. For nucleic acids, the length of comparison sequences will generally be at least 60 nucleotides, preferably at least 90 nucleotides, and more preferably at least 120 nucleotides.

By “high stringency conditions” is meant any set of conditions that are characterized by high temperature and low ionic strength and allow hybridization comparable with those resulting from the use of a DNA probe of at least 40 nucleotides in length, in a buffer containing 0.5 M NaHPO4, pH 7.2, 7% SDS, 1 mM EDTA, and 1% BSA (Fraction V), at a temperature of 65 C, or a buffer containing 48% formamide, 4.8×SSC, 0.2 M Tris-Cl, pH 7.6, 1× Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. See, e.g., F. Ausubel et al., Current Protocols in Molecular Biology, John Wiley & Sons, New York, N.Y., 1998, hereby incorporated by reference. Other features and advantages of the invention will be apparent from the following detailed description, and from the claims.

### BRIEF DESCRIPTION OF THE DRAWINGS

FIG. 1 is an amino acid sequence of a human intestinal trefoil factor (ITF; Accession No. BAA95531; SEQ ID NO: 1).

FIG. 2 is an amino acid sequence of a human pS2 protein (Accession No. NP_{—}003216; SEQ ID NO:2).

FIG. 3 is an amino acid sequence of human spasmolytic polypeptide (SP; Accession No. 1909187A; SEQ ID NO:3).

FIG. 4 is a cDNA sequence encoding a human intestinal trefoil factor (SEQ ID NO:4).

FIG. 5 is a cDNA sequence encoding a human pS2 protein (SEQ ID NO:5).

FIG. 6 is a cDNA sequence encoding a human spasmolytic polypeptide (SEQ ID NO:6).

FIG. 7 is the nucleotide sequence of a gene encoding human intestinal trefoil factor (locus 10280533:52117-55412; SEQ ID NO:7).

FIG. 8 is the nucleotide sequence of a gene encoding human pS2 protein (locus 10280533:16511-21132; SEQ ID NO:8).

FIG. 9 is the nucleotide sequence of a gene encoding human spasmolytic polypeptide (locus 10280533:957-5208; SEQ ID NO:9).

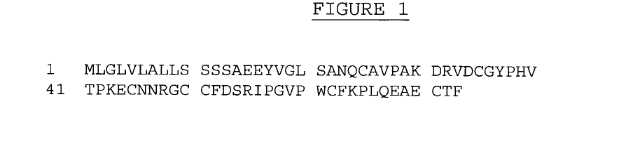

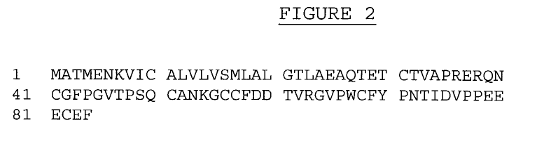

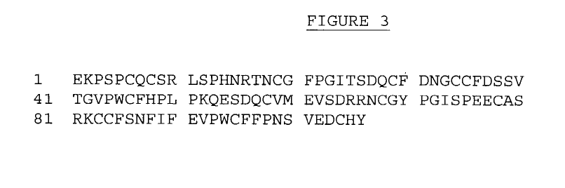

### DETAILED DESCRIPTION

The invention provides methods and compositions useful for the treatment of a wide range of lesions of the distal bowel. Lesions amenable to treatment using the methods and compositions of this invention include epithelial lesions of the anal sphincters, rectum, and colon, particularly the sigmoid colon and the descending colon. These lesions are treated by local application of intestinal trefoil peptides either alone or in combination with a second therapeutic agent and may be administered by any delivery device that is useful for delivering therapeutics to the distal bowel.

Pharmaceutical Preparations

Enemas

The enemas used to deliver the intestinal trefoil peptides of this invention are retention enemas, not evacuation enemas. Enemas, when administered in volumes of less than about 50 ml, deliver therapeutics to the rectum and sigmoid colon. However, enema volumes of about 150-250 ml can be used to deliver therapy to the descending, transverse and, in some cases, the ascending colon.

ITF-Containing Enema for Treatment of Ulcerative Colitis

| | |
|---|---|
| Intestinal trefoil factor | 750 mg |
| Sulfasalazine | 3 grams |
| Distilled water | 250 ml |

ITF-Containing Bulk Enema Suspension Suitable for Refrigeration

| | | |
|---|---|---|
| Intestinal trefoil factor | 5 | g/L |
| 5-aminosalicylic acid | 42 | g/L |
| NaH₂PO₄ | 0.4 | g/L |
| Na₂HPO₄ | 4.48 | g/L |
| NaCl | 9 | g/L |
| Sodium ascorbate | 0.5 | g/L |
| Tragacanth | 4 | g/L |
| Methylparaben | 2 | g/L |
| Propylparaben | 0.5 | g/L |
| Propylene glycol | 25 | ml/L |
| Distilled water | | |

Suppositories

Suppositories are solid dosage forms for insertion into the rectum for delivering medication to the rectum and sigmoid colon. Typically, after insertion, the suppository softens, melts, disperses, or dissolves in the lumenal fluid. Rectal suppositories for adults are usually about 2-5 grams each and tapered on both ends. Infant suppositories are usually about half the size of the adult formulations.

Either a fatty or a water soluble/water miscible suppository base can be used in the compositions of this invention. Suitable fatty bases include, for example, cocoa butter, theobroma oil, vegetable oils modified by esterification, hydrogenation, glycerinated gelatin and high molecular weight polyethylene glycols. Sustained release and/or prolonged contact of the therapeutics can be achieved by proper selection of a fatty suppository base material. Cocoa butter, for example, melts quickly at body temperature but is immiscible with body fluids, resulting in a prolonged but low level delivery of fat-soluble therapeutics to the affected sites. Alternatively, water soluble or water miscible bases (e.g., polyethylene glycols and glycol-surfactant mixtures) typically dissolve or disperse quickly, resulting in a rapid delivery of the therapeutic to the affected sites. An exemplary suppository formulation is provided below.

ITF-Containing Suppository Tablet

| Intestinal trefoil factor | 300 mg |
|---|---|
| Polyethylene glycol 1000 | 96% |
| Polyethylene glycol 4000 | 4% |

This formulation has a low-melting point and may require refrigeration to maintain in a solid state. Because the intestinal trefoil peptides are proteinaceous, refrigeration may be desirable. The low melting point of the formulation results in rapid suppository melting following insertion, resulting in greater patient comfort. If refrigeration is not possible, or if heat molding techniques are used, the amount of polyethylene glycol 4000 may be increased to achieve a sufficiently heat stable formulation.

In an alternative formulation, the intestinal trefoil peptides and/or other therapeutics can be encapsulated in bioerodable microspheres rather than being dissolved in the aqueous phase of the formulation. A wide variety of microencapsulation drug delivery systems have been developed and many share similar polymeric compositions as used for bioerodable films. Polymers commonly used in the formation of microspheres include, for example, methylacrylate polymers, poly-ε-caprolactone, poly(ε-caprolactone-Co-DL-lactic acid), poly(DL-lactic acid), poly(DL-lactic acid-Co-glycolic acid) and poly(ε-caprolactone-Co-glycolic acid) (see, for example, Pitt et al., J. Pharm. Sci., 68:1534, 1979).

Microspheres can be made by procedures well known in the art including spray drying, coacervation, and emulsification (see for example Davis et al. Microsphere and Drug Therapy, Elsevier, 1984; Benoit et al. Biodegradable Microspheres: Advances in Production Technologies, Chapter 3, Ed. Benita, S, Dekker, New York, 1996; Microencapsulation and Related Drug Processes, Ed. Deasy, Dekker, 1984, New York; U.S. Pat. No. 6,365,187). Preferably, the microspheres are bioadhesive or are prepared in formulations containing a bioadhesive excipient.

Other technical features of the intestinal trefoil peptide-containing solutions are easily modified to suit the specific pharmaceutical formulation and the clinical indication being treated. For example, the pH and osmolarity of the formulation may be adjusted to confer trefoil peptide stability, while minimizing gastrointestinal irritancy and sensitivity.

Ointments, Pastes, and Gels

Lesions of the epithelium of the anal sphincter and the surrounding skin, such as those resulting from trauma or hemorrhoids, are amenable to trefoil peptide therapy delivered as an ointment, paste, or gel. The viscous nature of these types of preparations allows for direct application into the wound site. Optionally, the wound site can be covered with a dressing to retain the trefoil peptide-containing composition, protect the lesion and/or absorb exudate. As discussed further below, these preparations are particularly useful to restore epithelial integrity following traumatic surgical procedures (e.g., hemorrhoidectomy). Such viscous formulations may also have a local barrier effect thereby reducing irritation and pain.

Mucoadhesives

A mucoadhesive excipient can be added to any of the previously described pharmaceutical compositions. The mucoadhesive formulations coat the lesioned area, resulting in retention of the intestinal trefoil peptide at the lesion site, providing protection, inhibiting irritation, and accelerating healing of inflamed or damaged tissue. Mucoadhesive formulations suitable for use in these pharmaceutical preparations are well known in the art (e.g., U.S. Pat. No. 5,458,879). Particularly useful mucoadhesives are hydrogels composed of about 0.05-20% of a water-soluble polymer such as, for example, poly(ethylene oxide), poly(ethylene glycol), poly(vinyl alcohol), poly(vinyl pyrrolidine), poly(acrylic acid), poly(hydroxy ethyl methacrylate), hydroxyethyl ethyl cellulose, hydroxy ethyl cellulose, chitosan, and mixtures thereof. These polymeric formulations can also contain a dispersant such as sodium carboxymethyl cellulose (0.5-5.0%).

Other preferred mucoadhesive excipients for liquid compositions are ones that allow the composition to be administered as a flowable liquid but will cause the composition to gel in the distal bowel, thereby providing a bioadhesive effect which acts to hold the therapeutic agents at the lesion site for an extended period of time. The anionic polysaccharides pectin and gellan are examples of materials which when formulated into a suitable composition will gel in the distal bowel, owing to the presence of cations in the mucosal fluids. The liquid compositions containing pectin or gellan will typically consist of 0.01-20% w/v of the pectin or gellan in water or an aqueous buffer system.

Other useful compositions which promote mucoadhesion and prolonged therapeutic retention in the distal bowel are colloidal dispersions containing 2-50% colloidal particles such as silica or titanium dioxide. Such formulations form as a flowable liquid with low viscosity suitable as an enema; however, the particles interact with glycoprotein, especially mucin, transforming the liquid into a viscous gel, providing effective mucoadhesion (e.g., U.S. Pat. Nos. 5,993,846 and 6,319,513).

Therapeutics Agents

Trefoil Peptides

The therapeutic intestinal trefoil peptide(s) are typically mammalian intestinal trefoil peptides. Preferably, human intestinal trefoil peptides are used; however, trefoil peptides from other species including rat, mouse, and non-human primate, may be used. Typically, the intestinal trefoil peptide is intestinal trefoil factor (ITF); however, spasmolytic polypeptide (SP), or pS2 are also useful.

The intestinal trefoil peptides are administered at 1-5000 mg per dose, preferably 5-2500 mg per dose, or more preferably 10-1500 mg per dose, depending on the nature and condition of the lesion being treated, the anticipated frequency and duration of therapy, and the type of pharmaceutical composition used to deliver the trefoil peptide. The intestinal trefoil peptides are typically administered 1-5 times per day.

Therapeutic Fragments of Intestinal Trefoil Factor (ITF)

We have also discovered that particular ITF fragments retain biological activity and may be substituted in any method or composition in which ITF is used. Methods and compositions containing ITF, in which these ITF fragments may be substituted, are described, for example, in U.S. Pat. Nos. 6,063,755 and 6,221,840, and U.S. patent application Ser. No. 10/131,363, filed Apr. 24, 2002, No. 60/317,657, filed Sep. 6, 2001, No. 60/327,673, filed Oct. 5, 2002, No. 60/333,836, filed Nov. 28, 2001, and No. 60/367,574, filed Mar. 26, 2002 (hereby incorporated by reference).

Particularly useful ITF fragments that retain biological activity include the polypeptide corresponding to amino acid residues 15-73 of SEQ ID NO: 1 (ITF₁₅₋₇₃) and amino acid residues 21-73 of SEQ ID NO: 1 (ITF₂₁₋₇₃). Other useful ITF fragments are formed following cleavage of the C-terminal phenylalanine residue (i.e., ITF₁₋₇₂, ITF₁₅₋₇₂, and ITF₂₁₋₇₂).

The biologically active ITF fragments of this invention can be produced using any appropriate method. For example, cDNA encoding the desired ITF fragment can be used with any method known in the art for producing recombinant proteins. Exemplary methods are provided herein. ITF fragments, particularly ITF₂₁₋₇₃, can be produced using a Pichia yeast expression system (see, for example, U.S. Pat. Nos. 4,882,279 and 5,122,465) transformed with a cDNA encoding long ITF species, such as the full length ITF (e.g., SEQ ID NO:4) or ITF₁₅₋₇₃, when the fermentation culture is maintained at pH ˜5.0.

Anti-Inflammatory Agents

Any suitable anti-inflammatory agent can be formulated with the trefoil peptide and employed using the method of this invention. Suitable anti-inflammatory agents include, but are not limited to non-steroidal anti-inflammatory drugs (e.g., ibuprofen, tacrolimus), cyclooxygenase-2-specific inhibitors such as rofecoxib (Vioxx®) and celecoxib (Celebrex®, topical glucocorticoid agents and specific cytokines directed at T lymphocyte function. Anti-inflammatory concentrations known to be effective following rectal administration can be used. For example, ibuprofen may be present in the composition at concentrations sufficient to deliver between 25-800 mg per day to the lesion. Corticosteroids may be co-formulated with a trefoil peptide at concentrations known to be effective for local rectal use.

Antimicrobial Agents

Any of the many known antimicrobial agents can be used in the compositions of the invention at concentrations generally used for these agents. Antimicrobial agents include antibacterials, antifungals, and antivirals.

Examples of antibacterial agents (antibiotics) include the penicillins (e.g., penicillin G, ampicillin, methicillin, oxacillin, and amoxicillin), the cephalosporins (e.g., cefadroxil, ceforanid, cefotaxime, and ceftriaxone), the tetracyclines (e.g., doxycycline, minocycline, and tetracycline), the aminoglycosides (e.g., amikacin, gentamycin, kanamycin, neomycin, streptomycin, and tobramycin), the macrolides (e.g., azithromycin, clarithromycin, and erythromycin), the fluoroquinolones (e.g., ciprofloxacin, lomefloxacin, and norfloxacin), and other antibiotics including chloramphenicol, clindamycin, cycloserine, isoniazid, rifampin, and vancomycin.

Antiviral agents are substances capable of destroying or suppressing the replication of viruses. Examples of anti-viral agents include 1,-D-ribofuranosyl-1,2,4-triazole-3 carboxamide, 9−>2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir.

Antifungal agents include both fungicidal and fungistatic agents such as, for example, amphotericin B, butylparaben, clindamycin, econaxole, fluconazole, flucytosine, griseofulvin, nystatin, and ketoconazole.

Analgesics and Anesthetics

Any of the commonly used topical analgesics can be used in the compositions of the invention. The analgesic is present in an amount such that there is provided to the distal bowel lesion a concentration of between one-half and five percent concentration for lidocaine (5-50 mg/ml in 20-40 ml per dose of liquid). Examples of other useful anesthetics include procaine, lidocaine, tetracaine, dibucaine, benzocaine, p-buthylaminobenzoic acid 2-(diethylamino) ethyl ester HCl, mepivacaine, piperocaine, and dyclonine.

Other analgesics include opioids such as, for example, morphine, codeine, hydrocodone, and oxycodone. Any of these analgesics may also be co-formulated with other compounds having analgesic or anti-inflammatory properties, such as acetaminophen, aspirin, and ibuprofen.

Steroids

Steroids may be used to treat lesions of the distal bowel. For example, ulcerative colitis may be treated using a paste preparation of triamcinolone (0.1%), hydrocortisone, fluticasone, budesonide, or beclomethasone.

5-Aminosalicylate Derivatives

5-aminosalicylate (5-ASA) derivatives are known to be useful for treating inflammatory bowel diseases such as Crohn's Disease and ulcerative colitis. Particularly useful 5-ASA derivatives include, for example, sulfasalazine, mesalamine, olsalazine, and balsalazide. Sulfasalazine is typically administered as a 3% enema, or orally in doses of 500-1000 mg. Mesalamine is normally administered as a one gram enema, daily for 3-6 weeks, or as a 500 mg suppository, 2-3 times per day for 3-6 weeks. Similar formulations may be prepared for any 5-ASA derivative.

Production of Intestinal Trefoil Peptides

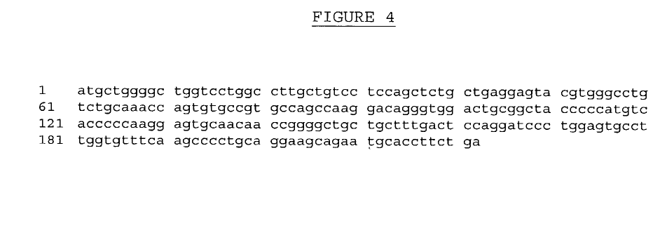

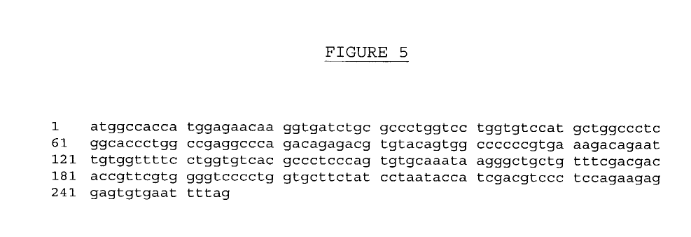

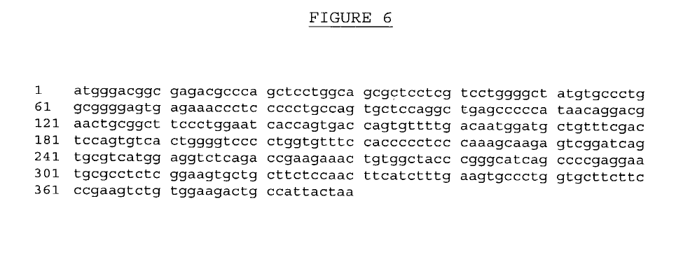

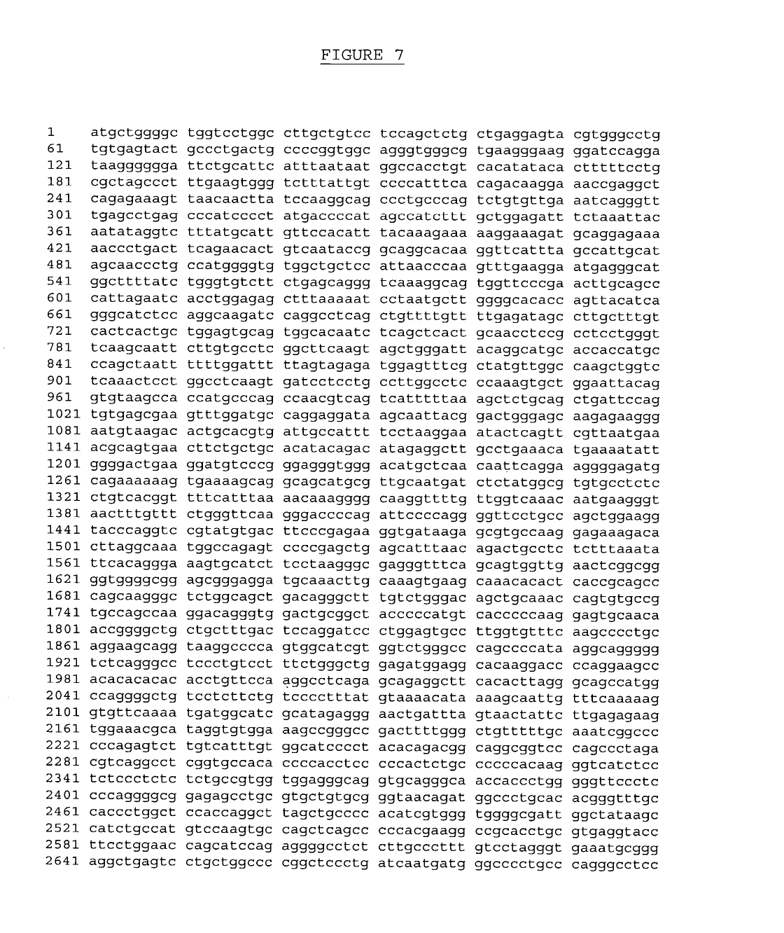

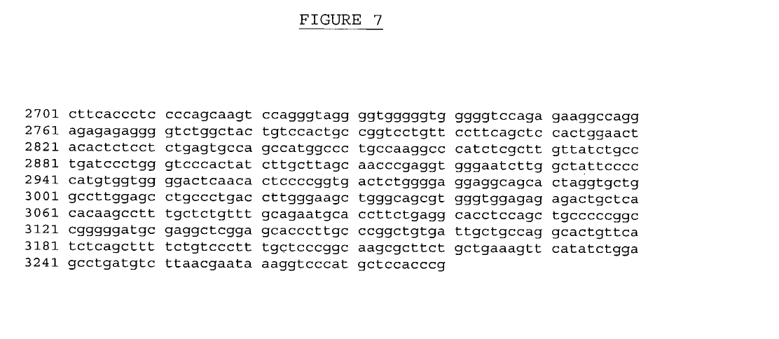

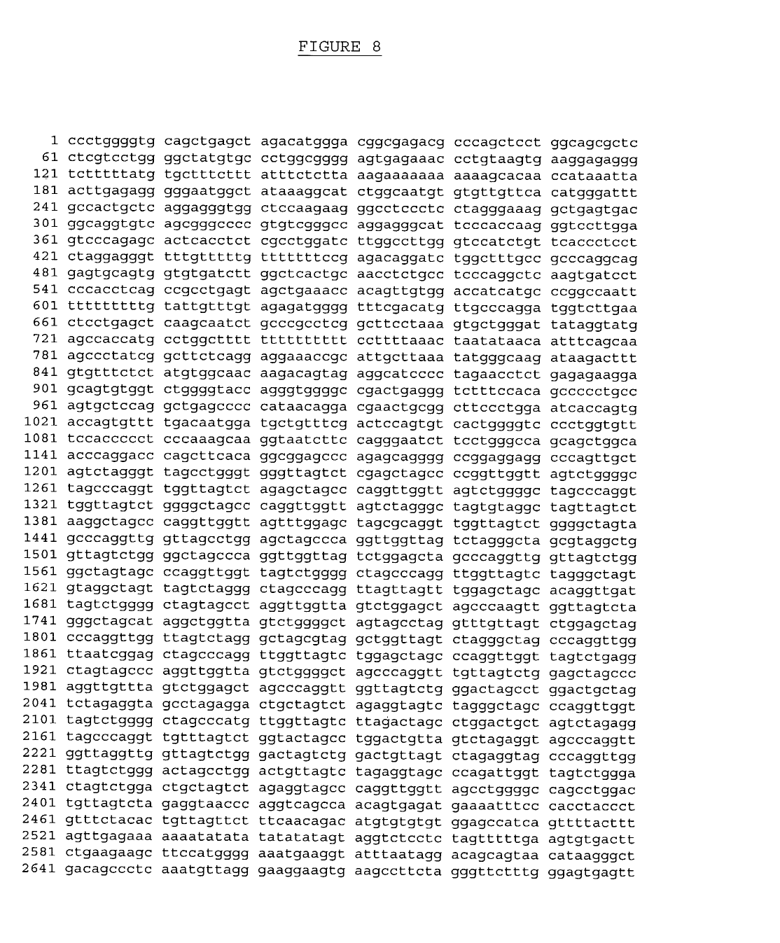

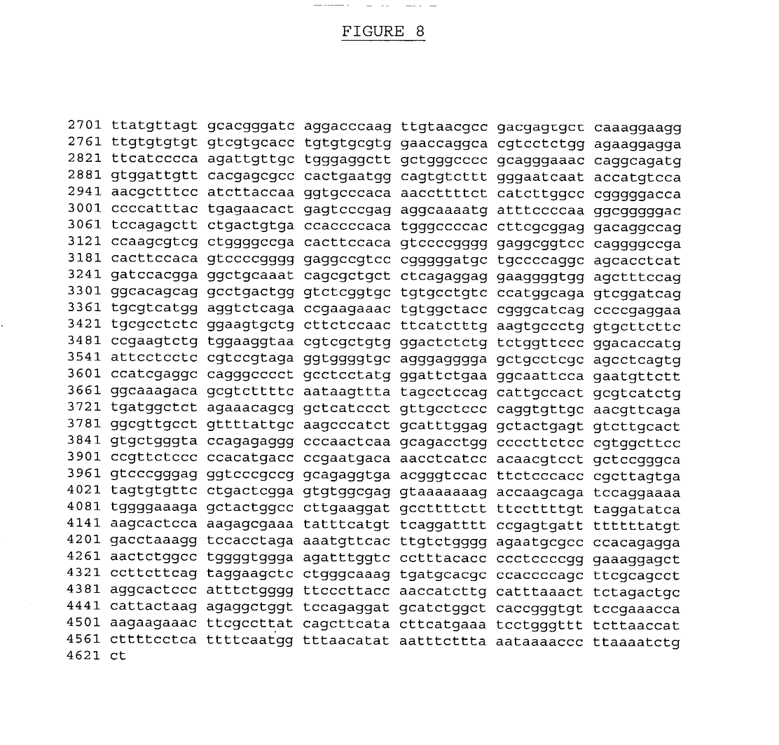

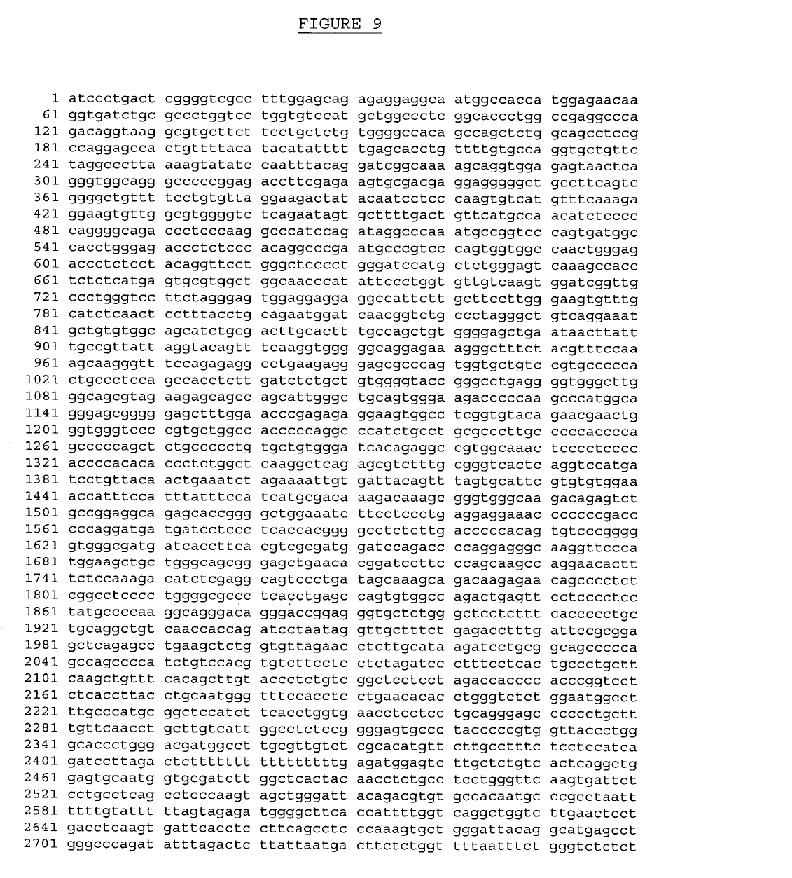

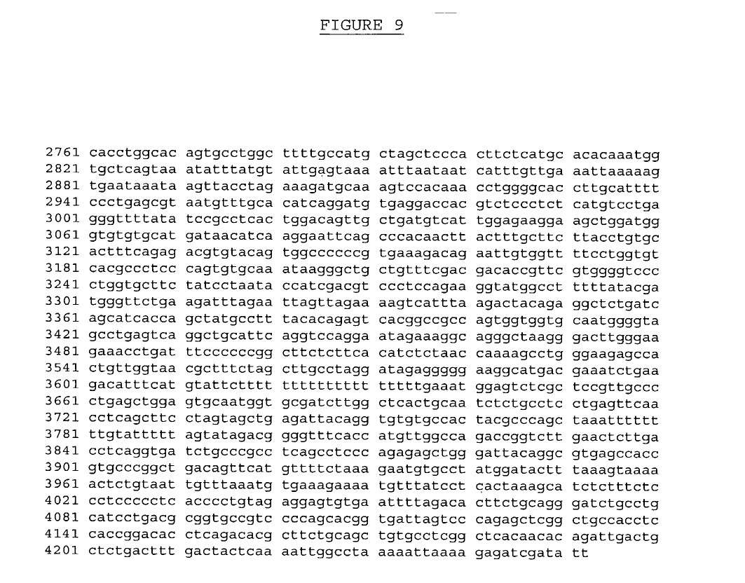

Intestinal trefoil peptides and fragments can be produced by any method known in the art for expression of recombinant proteins. Nucleic acids that encode trefoil peptides (e.g., human intestinal trefoil (factor (FIG. 4 and 7), human pS2 (FIG. 5 and 8), and human spasmolytic polypeptide (FIG. 6 and 9) or fragments thereof may be introduced into various cell types or cell-free systems for expression thereby allowing large-scale production, purification, and patient therapy.

Eukaryotic and prokaryotic trefoil peptide expression systems may be generated in which an intestinal trefoil peptide gene sequence is introduced into a plasmid or other vector, which is then used to transform living cells. Constructs in which the intestinal trefoil peptide cDNA contains the entire open reading frame inserted in the correct orientation into an expression plasmid may be used for protein expression. Prokaryotic and eukaryotic expression systems allow for the expression and recovery of intestinal trefoil peptide fusion proteins in which the trefoil peptide is covalently linked to a tag molecule which facilitates identification and/or purification. An enzymatic or chemical cleavage site can be engineered between the trefoil peptide and the tag molecule so that the tag can be removed following purification.

Typical expression vectors contain promoters that direct the synthesis of large amounts of mRNA corresponding to the inserted intestinal trefoil peptide nucleic acid in the plasmid-bearing cells. They may also include a eukaryotic or prokaryotic origin of replication sequence allowing for their autonomous replication within the host organism, sequences that encode genetic traits that allow vector-containing cells to be selected for in the presence of otherwise toxic drugs, and sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

Expression of foreign sequences in bacteria, such as Escherichia coli, requires the insertion of an intestinal trefoil peptide nucleic acid sequence into a bacterial expression vector. Such plasmid vectors contain several elements required for the propagation of the plasmid in bacteria, and for expression of the DNA inserted into the plasmid. Propagation of only plasmid-bearing bacteria is achieved by introducing, into the plasmid, selectable marker-encoding sequences that allow plasmid-bearing bacteria to grow in the presence of otherwise toxic drugs. The plasmid also contains a transcriptional promoter capable of producing large amounts of mRNA from the cloned gene. Such promoters may be (but are not necessarily) inducible promoters that initiate transcription upon induction. The plasmid also preferably contains a polylinker to simplify insertion of the gene in the correct orientation within the vector.

Mammalian cells can also be used to express a trefoil peptide. Stable or transient cell line clones can be made using intestinal trefoil peptide expression vectors to produce the trefoil peptides in a soluble (truncated and tagged) form. Appropriate cell lines include, for example, COS, HEK293T, CHO, or NIH cell lines.

Once the appropriate expression vectors are constructed, they are introduced into an appropriate host cell by transformation techniques, such as, but not limited to, calcium phosphate transfection, DEAE-dextran transfection, electroporation, microinjection, protoplast fusion, or liposome-mediated transfection. The host cells that are transfected with the vectors of this invention may include (but are not limited to) E. coli or other bacteria, yeast, fungi, insect cells (using, for example, baculoviral vectors for expression in SF9 insect cells), or cells derived from mice, humans, or other animals. In vitro expression of trefoil peptides, fusions, or polypeptide fragments encoded by cloned DNA may also be used. Those skilled in the art of molecular biology will understand that a wide variety of expression systems and purification systems may be used to produce recombinant trefoil peptides and fragments thereof. Some of these systems are described, for example, in Ausubel et al. (Current Protocols in Molecular Biology, John Wiley & Sons, New York, N.Y. 2000, hereby incorporated by reference).

Transgenic plants, plant cells and algae are also particularly useful for generating recombinant intestinal trefoil peptides for use in the methods and compositions of the invention. For example, transgenic tobacco plants or cultured transgenic tobacco plant cells expressing an intestinal trefoil peptide can be created using techniques known in the art (see, for example, U.S. Pat. Nos. 5,202,422 and 6,140,075). Transgenic algae expression systems can also be used to produce recombinant intestinal trefoil peptides (see, for example, Chen et al., Curr. Genet. 39:365-370, 2001).

Once a recombinant protein is expressed, it can be isolated from cell lysates using protein purification techniques such as affinity chromatography. Once isolated, the recombinant protein can, if desired, be purified further by e.g., high performance liquid chromatography (HPLC; e.g., see Fisher, Laboratory Techniques In Biochemistry And Molecular Biology, Work and Burdon, Eds., Elsevier, 1980).

Polypeptides of the invention, particularly short intestinal trefoil peptide fragments can also be produced by chemical synthesis using, for example, Merrifield solid phase synthesis, solution phase synthesis, or a combination of both (see, for example, the methods described in Solid Phase Peptide Synthesis, 2nd ed., 1984, The Pierce Chemical Co., Rockford, Ill). Optionally, peptide fragments are then be condensed by standard peptide assembly chemistry.

Dosages

All of the therapeutic agents employed in the topical compositions of the present invention, including the trefoil peptide component, can be used in the dose ranges currently known and used for these agents. The following are illustrative examples of dose ranges for the active ingredients of the compositions of the invention. Different concentrations of either the trefoil peptide or the other agents may be employed depending on the clinical condition of the patient, the goal of therapy (treatment or prophylaxis), and anticipated duration or severity of the damage for which the agent is being given. Additional considerations in dose selection include: disease etiology, patient age (pediatric, adult, geriatric), general health and comorbidity.

The following examples are intended to illustrate the principle of the present invention and circumstances when trefoil peptide therapy is indicated. The following examples are not intended to be limiting.

### EXAMPLE 1

### Methods for Treating Proctitis, Enteritis, or Mucositis Associated with Antineoplastic Therapy

Antineoplastic therapy, including chemotherapy and radiotherapy, can damage the intestinal epithelium, resulting in proctitis, enteritis, or mucositis. Damage to the intestinal mucosa is especially prevalent when wide area radiotherapy is delivered to the abdomen for the treatment of, for example, colorectal, cervical, uterine, ovarian, or prostate cancer. Therapeutic amounts of trefoil peptides can be administered either previous to, concurrent with, or subsequent to antineoplastic therapy and can be delivered, for example, as an enema or by rectal suppository. Trefoil peptide therapy that follows antineoplastic therapy should begin within the first 14 days after the final antineoplastic treatment, preferably within the first 7 days, more preferably within the 3 days, even more preferably within the first day and most preferably, immediately following said final antineoplastic treatment.

Alternatively, intestinal trefoil peptide therapy can be administered concurrent to the antineoplastic therapy regime. Effective concurrent therapy consists of trefoil peptide administration within 12 hours, 6 hours, 3 hours, or simultaneously with every antineoplastic treatment.

Intestinal trefoil peptide therapy can also begin prior to initiation of the antineoplastic therapy regime. Pretreatment with a trefoil peptide is prophylactic, thereby mitigating the loss of intestinal epithelial cells which normally occurs as a consequence of cancer therapy. Trefoil peptide therapy normally begins 14 days, 7 days, 3 days, or 1 day prior to beginning an antineoplastic therapy.

The most preferred embodiment of the present method consists of continuous trefoil peptide therapy which is begins with a pretreatment phase, prior to the initiation of antineoplastic therapy, and continues concurrently and subsequently to the cancer therapy.

Rectal administration of a therapeutically effective amount of a trefoil peptide composition using the method of the present invention is done between once and six times each day, as clinically indicated. Further, clinical indications may warrant supplementation of rectal trefoil peptide therapy with oral administration of the same or another trefoil peptide, or even another intestinal healing agent. Likewise, clinical indications may necessitate the addition of one or more therapeutic agents, for example, antimicrobials, analgesics, and anti-inflammatories. Additional medicaments can be co-formulated with the trefoil peptides, or may be administered separately.

### EXAMPLE 2

### Methods for Treating Mucosal Damage Caused by a Physical Injury

Compositions containing a trefoil peptide, are used to lessen complications and speed healing of the wound created by the surgical procedure or other traumatic injury. These injuries can be caused by procedures like, for example, a biopsy, a hemorrhoidectomy, or a bowel resection. An aqueous based enema, gel, paste, or suppository, as described above, is applied immediately following the procedure or injury. Alternatively, a more concentrated medication can be directly applied to the wound via a pledget with a stick applicator. Preferably, the trefoil peptide is applied immediately following the surgical procedure and then every six hours until epithelial healing is complete. Treatment with a trefoil peptide prior to surgical intervention can speed post-operative mucosal healing.

### EXAMPLE 3

### Methods for Treating Crohn's Disease

Crohn's Disease can be treated using a combination of rectally administered ITF and intravenously administered infliximab (Remicade®)). Infliximab is administered by a single intravenous infusion of 5 mg/kg, over about 2 hours. The patient self-administers one suppository, containing one gram of ITF, every second day, beginning 4 days prior to infliximab treatment. ITF therapy, using the suppositories, is continued every second day for six weeks, or as clinically indicated. Patients having severe Crohn's disease may require additional infliximab therapy two weeks and six weeks after the first infusion.

Other Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for treating or preventing a lesion of the distal bowel of a mammal comprising administering to the rectum of said mammal a therapeutically effective amount of an intestinal trefoil peptide.

2. The method of claim 1, wherein said intestinal trefoil peptide is spasmolytic polypeptide, pS2, or intestinal trefoil factor.

3. The method of claim 2, wherein said intestinal trefoil peptide is intestinal trefoil factor.

4. The method of claim 1, wherein said intestinal trefoil peptide is ITF₁₅₋₇₃, ITF₂₁₋₇₃, ITF₁₋₇₂, ITF₁₅₋₇₂, or ITF₂₁₋₇₂.

5. The method of claim 1, wherein said mammal is a human.

6. The method of claim 1, wherein said lesion is enteritis or proctitis.

7. The method of claim 1, wherein said lesion is caused by Crohn's Disease.

8. The method of claim 1, wherein said lesion is caused by ulcerative colitis.

9. The method of claim 1, wherein said lesion is caused by antineoplastic therapy.

10. The method of claim 9, wherein said antineoplastic therapy is radiation therapy.

11. The method of claim 9, wherein said antineoplastic therapy is chemotherapy.

12. The method of claim 1, wherein said lesion is the result of a hemorrhoidectomy.

13. The method of claim 1, wherein said lesion is the result of a biopsy procedure or surgical intervention.

14. The method of claim 13, wherein said lesion is the result of tumor resection.

15. The method of claim 1, wherein said lesion is caused by a bacterial, viral, or fungal infection.

16. The method of claim 1, further comprising administering to said mammal a second therapeutic.

17. The method of claim 16, wherein said second therapeutic agent is an anti-inflammatory agent.

18. The method of claim 16, wherein said second therapeutic agent is rofecoxib or celecoxib.

19. The method of claim 16, wherein said second therapeutic agent is an antibacterial agent.

20. The method of claim 19, wherein said antibacterial agent is a penicillin, a cephalosporin, a tetracycline, or an aminoglycoside.

21. The method of claim 16, wherein said second therapeutic agent is an anti-fungal agent.

22. The method of claim 21, wherein said anti-fungal agent is nystatin or Amphotericin B.

23. The method of claim 16, wherein said second therapeutic agent is an anti-viral agent.

24. The method of claim 23, wherein said anti-viral agent is acyclovir.

25. The method of claim 16, wherein said second therapeutic agent is an analgesic.

26. The method of claim 25, wherein said analgesic is lidocaine or benzocaine.

27. The method of claim 16, wherein said second therapeutic agent is a steroid.

28. The method of claim 27, wherein said steroid is triamcinolone, budesonide, or hydrocortisone.

29. The method of claim 16, wherein said second therapeutic is sulfasalazine, mesalamine, olsalazine, balsalazide, or metronidazole.

30. The method of claim 16, wherein said second therapeutic is infliximab.

31. The method of claim 16, wherein said trefoil peptide and said second therapeutic are administered in the same formulation.

32. The method of claim 16, wherein said trefoil peptide and said second therapeutic are administered in different formulations.

33. The method of claim 32, wherein said trefoil peptide and said second therapeutic are administered within 14 days of each other.

34. The method of claim 33, wherein said trefoil peptide and said second therapeutic are administered within 24 hours of each other.

35. The method of claim 9, wherein said intestinal trefoil peptide is administered within 14 days of said antineoplastic therapy.

36. The method of claim 35, wherein said intestinal trefoil peptide is administered within 24 hours of said antineoplastic therapy.

37. A pharmaceutical composition suitable for therapeutic delivery to the rectum of a mammal, said composition comprising an intestinal trefoil peptide and a pharmaceutically acceptable carrier.

38. The composition of claim 37, wherein said intestinal trefoil peptide is spasmolytic polypeptide, pS2, or intestinal trefoil factor.

39. The composition of claim 38, wherein said intestinal trefoil peptide is intestinal trefoil factor.

40. The composition of claim 37, wherein said intestinal trefoil peptide is ITF₁₅₋₇₃, ITF₂₁₋₇₃, ITF₁₋₇₂, ITF₁₅₋₇₂, or ITF₂₁₋₇₂.

41. The composition of claim 37, wherein said composition is a suppository.

42. The composition of claim 37, wherein said composition is an enema.

43. The composition of claim 37, wherein said composition comprises microspheres.

44. The composition of claim 37, wherein said composition further comprises a mucoadhesive agent.

45. The composition of claim 37, wherein said composition further comprises a second therapeutic agent.

46. The composition of claim 45, wherein said second therapeutic agent is an anti-inflammatory agent.

47. The composition of claim 45, wherein said second therapeutic agent is an antibacterial agent.

48. The composition of claim 47, wherein said antibacterial agent is a penicillin, a cephalosporin, a tetracycline, or an aminoglycoside.

49. The composition of claim 45, wherein said second therapeutic agent is an anti-fungal agent.

50. The composition of claim 49, wherein said anti-fungal agent is nystatin or Amphotericin B.

51. The composition of claim 45, wherein said second therapeutic agent is an anti-viral agent.

52. The composition of claim 51, wherein said anti-viral agent is acyclovir.

53. The composition of claim 45, wherein said second therapeutic agent is an analgesic.

54. The composition of claim 53, wherein said analgesic is lidocaine or benzocaine.

55. The composition of claim 45, wherein said second therapeutic agent is a steroid.

56. The composition of claim 55, wherein said steroid is triamcinolone, budesonide, or hydrocortisone.

57. The composition of claim 45, wherein said second therapeutic is sulfasalazine, mesalamine, olsalazine, or balsalazide.

58. The composition of claim 45, wherein said second therapeutic is metronidazole.
